(19) United States
(12) Patent Application Publication
(10) Pub. No.: US 2025/0049451 A1
(43) Pub. Date: Feb. 13, 2025

(54) ASPIRATION CATHETER

(71) Applicants: Shashvat M. Desai, Phoenix, AZ (US); Ashutosh P. Jadhav, Paradise Valley, AZ (US); Kunakorn Atchaneeyasakul, Renton, WA (US); GRAVITY MEDICAL TECHNOLOGY, INC., Carlsbad, CA (US)

(72) Inventors: Shashvat M. Desai, Phoenix, AZ (US); Ashutosh P. Jadhav, Paradise Valley, AZ (US); Kunakorn Atchaneeyasakul, Renton, WA (US)

(21) Appl. No.: 18/720,934

(22) PCT Filed: Dec. 19, 2022
(86) PCT No.: PCT/US2022/053383

§ 371 Date: Jun. 17, 2024

Related U.S. Application Data
(60) Provisional application No. 63/291,703, filed on Dec. 20, 2021.

(51) Int. Cl. A61B 17/22; A61M 25/01

(52) U.S. Cl. CPC A61B 17/22; A61M 25/0147; A61B 2017/22079

(57) ABSTRACT

A device for aspirating a thrombus has an elongate shaft and a housing with an actuator coupled to the shaft. Pull wire lumens are disposed in a side wall of the elongate shaft and they have one end coupled to an actuator, and the opposite end coupled to the distal portion of the elongate shaft. Actuation of the actuator in a first direction steers the distal portion of the elongate shaft into a first configuration or changes a size or a shape of the lumen in the elongate shaft. Actuation of the actuator in the opposite direction releases tension in the pull wires to steer the elongate shaft into a second configuration or further changes the size or shape of the lumen.

### CLAIM OF PRIORITY

The present application is an International PCT patent application that claims the benefit of U.S. Provisional Patent Application No. 63/291,703 (Attorney Docket No. 5543.002PRV) filed on Dec. 20, 2021; the entire contents of which are incorporated herein by reference.

### BACKGROUND

Thrombus may build up in the arteries of a patient in sufficient quantity that the vessel becomes occluded resulting in insufficient blood flow to tissue supplied by the occluded vessel. This can cause a patient to experience a number of complications including death. Various drug therapies, surgical approaches or minimally invasive treatments exist for removing the thrombus from the vessel thereby restoring blood flow.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a schematic illustration of an example of an aspiration catheter.

FIGS. 2A-2G illustrate examples of actuator configurations.

FIG. 3A shows the elongate shaft in FIG. 1.

FIGS. 3B show a cross-section of the elongate shaft in FIG. 3A.

FIG. 4A shows a cross-section taken of the distal portion of the aspiration catheter in FIG. 1.

FIG. 4B show the construction of the distal portion of the aspiration catheter in FIG. 1.

FIGS. 5A1-5D2 show various distal lumen and distal elongate shaft sizes and shapes for the aspiration catheter of FIG. 1.

FIG. 6A shows and example of how a controller may be used to control actuation of the aspiration catheter in FIG. 1.

FIG. 6B shows an example of an actuator that may be used in FIG. 6A

FIGS. 7A-7B show an example of a method of removing a thrombus from a patient with an aspiration catheter.

FIGS. 7C-7D illustrates aspiration of thrombus using a conventional catheter compared to using an example of a catheter disclosed herein.

### DETAILED DESCRIPTION

Thrombus may build up in the arteries of a patient in sufficient quantity that the vessel becomes occluded resulting in insufficient blood flow to tissue supplied by the occluded vessel. This can cause a number of complications including patient death. Various drug therapies such as thrombolytics may be used to dissolve clots and surgical or other minimally invasive treatments exist for removing the thrombus from the vessel thereby restoring blood flow. Each of these treatments have advantages and disadvantages. For example, thrombolytic drugs can take a long period of time to dissolve a clot, may increase the risk of bleeding inside the brain, and can be costly. Surgery can also be costly and may require a long recovery period as well as result in unwanted scarring. Minimally invasive procedures are promising but cannot always reach distal regions in the vessels, navigate tortuous vessels or they may not be able to retrieve all the thrombus material. Further, minimally invasive devices, such as an aspiration catheter, may tend to have a fixed size and shape of the distal lumen engaging the thrombus. There is a need for personalized or individualized and customizable size and shape of the distal lumen of an aspiration catheter. Therefore, it would be desirable to provide improved devices, systems, and methods for removing thrombus (also referred to herein as a blood clot or clot) from a patient.

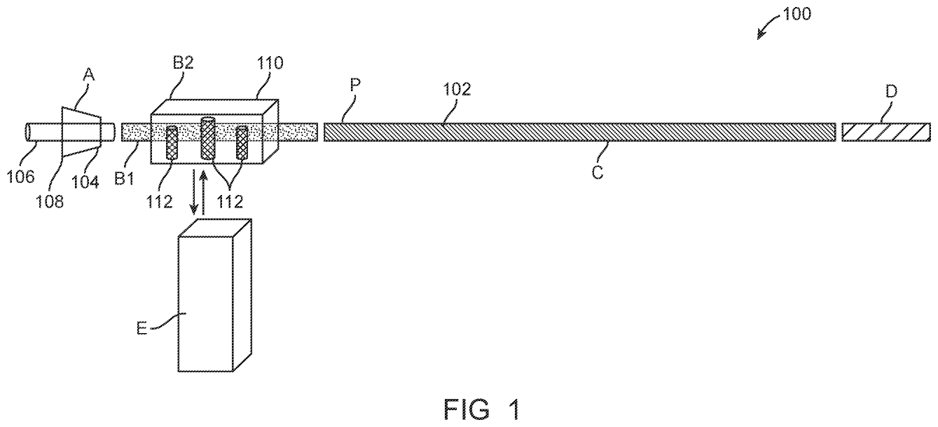

FIG. 1 shows a schematic illustration of an example of an aspiration catheter 100 that can be used to aspirate a clot from a patient's blood vessel such as an artery. The artery may be anywhere in the body, such as in the limbs or the brain. The catheter 100 may include an elongate shaft 102 having a proximal portion P and a distal portion D. The catheter may be any length in order to reach the target treatment region, but in some examples is approximately 130-145 cm long. The proximal portion P is closest to the operator and a portion may remain outside the patient. Opposite the proximal portion, the catheter may have a distal portion D which is furthest away from the operator and is generally disposed in the patient's body and has the working end which aspirates thrombus from the vessel.

Section A of the aspiration catheter 100 is the part of the proximal section P which remains outside the patient's body and may include a connector 104 such as a Luer connector, a barbed connector, or any other connector. The connector 104 allows the proximal end 106 of the catheter to be easily coupled and uncoupled from another device such as a vacuum pump, tubing, a syringe, or any other device. Also, the connector 104 may include an internal taper that allows a guidewire to be easily fed into the connector and into the aspiration catheter lumen. The connector 104 may also have wings 108 that extend laterally outward and away from the connector 104 for easy grasping/manipulation by an operator. The connector 104 may have other features on it such as holes in the connector or wings or a suture connector so that the connector 104 may be attached with tape, sutures, or any other technique to the patient to anchor the proximal end of the aspiration catheter.

Sections B1 and B2 are also part of the proximal portion P of the aspiration catheter 100 and typically remain outside the patient's body.

Section B1 is a proximal portion of the elongate shaft 102 which can be coupled to the connector in section A. Section B1 may have one or more lumens passing through it such as a central lumen for aspiration of the thrombus or for passing a guidewire through. The elongate shaft 102 may be braided in this section as will be discussed in greater detail below. Additionally, there may be other lumens in the walls of the elongate shaft in section B1 used to house pull wires as will described below.

Section B2 has a housing 110 that is coupled to the proximal portion P of elongate shaft 102. The housing may be an ergonomically designed handle that fits comfortably in an operator's hand during use and also facilitates easy actuation of the actuator on the handle during use. The elongate shaft 102 passes through the housing 110 and is coupled to one or more actuators 112 on the housing. In this example, three actuators 112 are illustrated but this may be less or more. A series of pull wires (not shown in this view) are coupled to the actuators and the pull wires extend through lumens in the wall of the elongate shaft toward the distal end of elongate shaft where their distal ends are coupled to an expandable distal end. Actuation of the various actuators controls steering of the distal end of the aspiration catheter as well as controlling the lumen size and shape at the distal end of the aspiration catheter. The actuators may be levers, slides, thumbwheels, motors, or any other mechanism that allows tensioning and de-tensioning of the pull wires.

Section C is the elongate shaft 102 of the aspiration catheter 100. It may a polymer tube, or another example of the catheter may be formed from an inner coiled tube or a polymer tube and an outer layer of braiding to provide desired material properties (e.g. stiffness, torqueability, pushability, etc.). The pull wires may be disposed in the annular space between the inner polymer tube and outer braiding, or through lumens in the wall of the elongate shaft. In some examples, a separate lumen is formed for each pull wire by passing a cuff or tubular liner from the proximal end of the elongate shaft to the distal end of the elongate shaft, between the polymer tube inner layer and the braided outer layer. The cuff or liner may be made from a low friction polymer such as PTFE (polytetrafluoroethylene) or any other material known in the art. There may be any number of pull wire lumens such as 1, 2, 3, 4, 5, 6, 7, 8, 9, 10, or more and the lumens may be spaced evenly around the circumference of the elongate shaft or in any desired pattern. The elongate shaft generally has a constant diameter and substantially linear shape in its unbiased state but diameter may change when the pull wires are actuated and the elongate shaft may have one or more curves when the pull wires are actuated.

Section D is the distal portion of the aspiration catheter 100 and generally this portion is disposed in the patient. It includes the working end that aspirates the thrombus from the patient. The distal portion may be any length but in this example is approximately the distal 3 cm of the catheter. The distal portion may be steered to curve the distal portion so that the distal end of the catheter may be moved adjacent to thrombotic material to be aspirated. Additionally, the central lumen of the elongate shaft has a distal aperture on the distal tip of the aspiration catheter and the size and shape of the distal aperture of the central lumen may also be adjusted as desired. Actuation of the actuators in the housing apply tension or release tension from the pull wires which steer the distal end of the elongate shaft and/or change the distal aperture lumen size and shape. The distal portion of the aspiration catheter may include plates coupled together with a flexible mesh to allow steering and size changes as will be discussed in more detail below.

Section E is an optional controller which may be releasably coupled to the housing B2. The controller may include motors, pistons or other motorized or actuatable components which can be coupled to the housing to move the actuators on the housing. The controller may also include a processor that can determine a desired distal catheter shape and/or distal aperture lumen size and shape to form based on an image (e.g. CT scan or angiogram) of the treatment site. Thus, based on the image of the treatment site, the processor determines the optimal size and shape of the distal portion of the aspiration catheter and the controller then actuates the actuators on the housing to move the pull wires to form the desired shape and size. At the end of the procedure, the aspiration catheter may be a disposable that is a single use device and may be thrown away after completion of a procedure, while the controller may be uncoupled from the housing and reused for another procedure with a new aspiration catheter after appropriate cleaning and sanitization or sterilization.

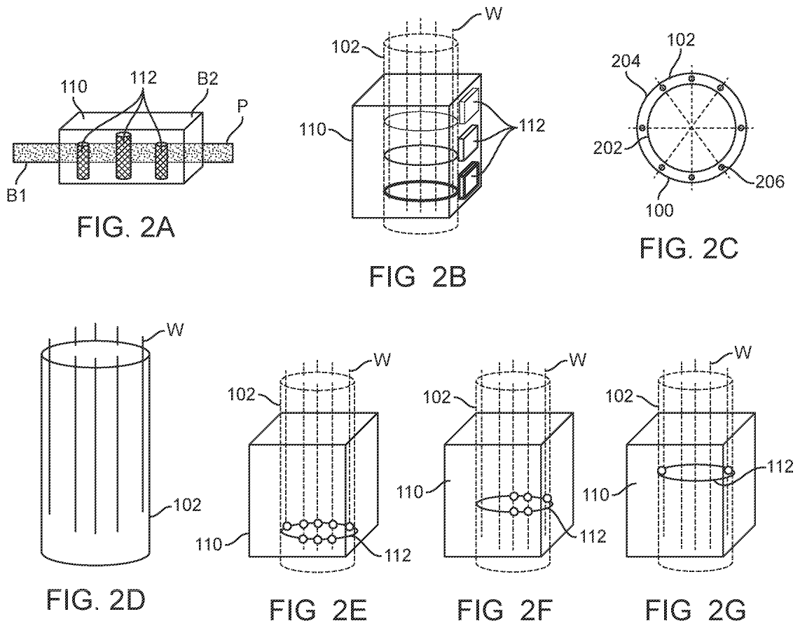

FIGS. 2A-2G illustrate examples of actuator configurations that may be used in the housing.

FIG. 2A shows the proximal portion P of the aspiration catheter 100 seen in FIG. 1 with housing 110. Here, the housing 110 has an actuator which includes three actuators 112 which control various groups of pull wires to effect change in the distal tip curvature by steering and/or by increasing or decreasing the distal aperture lumen size and changing lumen shape.

FIG. 2B schematically show the three actuators 112 (distal-most, proximal-most, and intermediate actuator disposed in between the proximal-most and distal-most actuators). Here, the distal-most actuator is coupled to several pull wires W (longitudinally oriented lines in phantom) to control steering and lumen size and shape of the catheter. The intermediate actuator is coupled to another group of pull wires W to steer or control lumen size and shape differently, and the proximal-most actuator is coupled to yet another group of pull wires W to steer or control lumen size and shape differently. As previously discussed, the actuators 112 may be any type of actuator known in the art such as levers, sliders, knobs, thumb wheels, motors, etc. The pull wires may be disposed individually in separate lumens disposed in a wall of the catheter, or the pull wires may be disposed in the central catheter lumen, or they may run along the outside of the catheter outer surface. Actuation of the actuators 112 will either apply tension to one or more pull wires W or release tension in one or more pull wires. The distal ends of the pull wires are coupled to a distal portion of the catheter as will be discussed in greater detail below.

FIGS. 2C shows a cross-section of the elongate shaft 102 of catheter 100 shown in FIG. 1, with the inner-most coil or polymer tube layer 202 and outer braided layer 204. The annular space in between the inner and outer layers includes a plurality of lumens 206 sized to receive the pull wires. The lumens may optionally include a cuff or liner such as Teflon® (polytetrafluoroethylene) to reduce friction as the pull wires are slidably advanced or retracted through the pull wire lumens 206. In this example, there are four lumens spaced apart 90 degree and they are located at the 12 o'clock, 3 o'clock, 6 o'clock and 9 o'clock positions. Two additional lumens are located on either side of the 12 o'clock lumen and they are displaced thirty degrees away from the 12 o'clock lumen. Thus, the two lumens which are displaced 30 degrees away from the 12 o'clock lumen and they are also 60 degrees away from the respective 3 o'clock and 9 o'clock lumens. Two additional lumens are also disposed 30 degrees on either side of the 6 o'clock lumen so that they are 60 degrees offset from the respective 3 o'clock and 6 o'clock lumens. Therefore, the pull wires will have similar orientations once disposed in their respective lumens. This configuration is not intended to be limiting and any other arrangement of the pull wires and lumens is possible. By adjusting the pull wire position circumferentially around the elongate shaft will allow different distal tip configurations to be achieved upon actuation of the pull wires with the actuators on the handle.

FIG. 2D shows the pull wires W running through the lumens in the elongate shaft 102. In this example, there are eight pull wires but this may be increased or decreased as needed. The pull wires extend from the actuator to the distal portion of the elongate shaft where the pull wires terminate and are coupled to the steering and lumen sizing components in the distal end of the elongate shaft. One or more pull wires may be coupled to an intermediate portion or the proximal portion of the elongate shaft (the intermediate portion is disposed between the proximal portion and the distal portion of the catheter) when it is desired to be able to control curvature of the intermediate portion or the proximal portion of the elongate shaft.

FIG. 2E shows an example of the proximal-most actuator 112 on housing 110 coupled to elongate shaft 110, where all eight pull wires W are coupled to the proximal-most actuator. Actuation of the proximal-most actuator will then move all eight pull wires proximally when tension is applied and distally when tension is released, which will expand or collapse the distal lumen size as will described in more detail below.

FIG. 2F shows an example of the intermediate actuator 112 where five pull wires W are coupled to the actuator. Here the five pull wires may include the 3 o'clock, 6 o'clock, and 12 o'clock pull wires as well as the pull wire between the 12 o'clock and 3 o'clock pull wire and the pull wire between the 3 o'clock and 6 o'clock pull wire (the pull wire that is displaced thirty degrees away from the 12 o'clock position clockwise, and the pull wire that is displaced thirty degrees from the 6 o'clock position counter clockwise). Actuation of the intermediate actuator 112 will steer the distal end of the aspiration catheter to one side (laterally toward the 3 o'clock position) without changing the aspiration lumen diameter. Additional details about this are disclosed below.

FIG. 2G shows an example of the distal-most actuator 112 where two pull wires W are coupled to the actuator 112. Here, the two pull wires include the 3 o'clock and 9 o'clock pull wires (or any other pair separated by 180 degrees). Actuation of the distal-most actuator 112 will change the lumen shape from a round lumen to an oval shaped lumen increasing lumen size along the major axis and decreasing lumen size along the minor axis of the oval. The distal portion of the elongate shaft is not steered in this configuration. Additional details about this are disclosed below.

Other actuator configurations are possible in order to control the lumen size and shape differently or the elongate shaft steering. For example, the pull wires in FIG. 2F may be coupled to an actuator to control the lumen and shaft size/shape opposite of the example in FIG. 2F resulting in a mirror image of shaped catheter tip. Or the example in FIG. 2G may be modified by altering pull wires by 90 degrees to change the orientation of the oval by 90 degrees so that the minor axis of the oval becomes the major axis of the oval and similarly the major axis of the oval becomes the minor axis of the oval. Any number of pull wire combinations are possible and they are not limited by the examples disclosed herein.

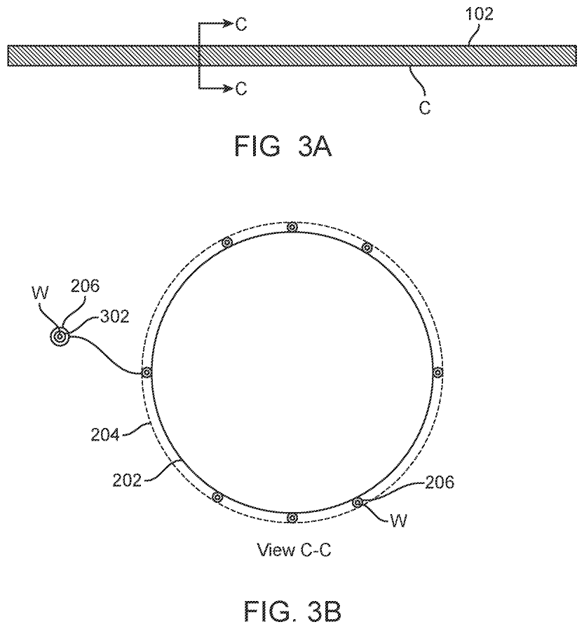

FIG. 3A shows the elongate shaft C (also referred to as element 102) that forms a portion of the aspiration catheter 100 in FIG. 1. The elongate shaft 102 is generally an elongated cylindrically shaped tube having a central lumen (not shown) disposed therein. The central lumen may be used to receive a guidewire during delivery to a target treatment area or for aspiration of thrombus, or for irrigation with a fluid, or any other purpose. The tube may be formed in any number of ways but in this example, includes an inner layer which is a helically coiled wire with a braided tube disposed over the coil. The two layers may be bonded together, encapsulated in a polymer or coupled together using other techniques known in the art. The annular space between the coiled wire and the braided tube may include one or more lumens for housing the pull wires or they may be disposed in a wall of the inner coil or a wall of the outer braided tube. The pull wire lumens may include a low friction cuff or liner as previously described above.

FIG. 3B shows a cross-section of elongate shaft C taken along the line C-C in FIG. 3A. This view clearly shows the outer braided layer 204 and the inner coil layer 202. Also the pull wire lumens 206 are shown in the annular space between the inner and outer layers 202, 204. In this example, there are eight pull wire lumens 206. Four are disposed ninety degrees apart from one another at the 12 o'clock, 3 o'clock, 6 o'clock, and 9 o'clock positions. Two pull wire lumens are offset 30 degrees from either side of the 12 o'clock lumen, and two additional pull wires are off set 30 degrees from either side of the 6 o'clock lumen. Thus, the pull wire lumen positions match the pull wire locations previously discussed above. The pull wire lumens 206 are optionally lined with a low friction liner 302 or cuff to facilitate slidable movement of the pull wires W through the pull wire lumens. In alternative examples, the pull wire lumens 206 may or may not include the liner or cuff and optionally may include a lubricant to reduce friction for the pull wires.

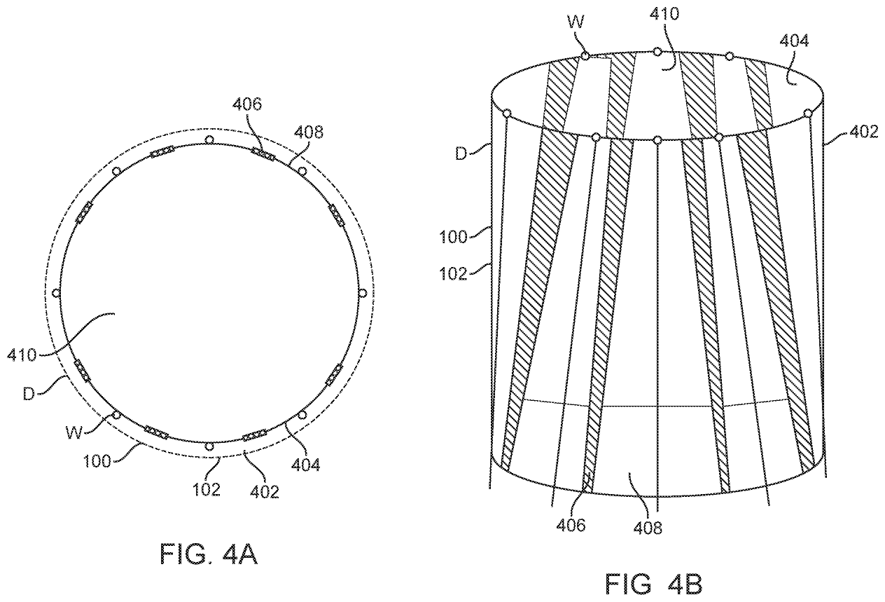

FIG. 4A shows a cross-section taken of the distal portion D of the aspiration catheter 100 shown in FIG. 1. The length of this section may be any length but in this example is approximately 3 cm long. The cross-section is taken in a plane orthogonal to the longitudinal axis of the aspiration catheter elongate shaft 102 in the distal portion. Here, the outer layer 402 is a flexible braided tubing and the inner layer 404 is an adjustment layer that is formed from a plurality of plates 408 that are coupled together with an elastic mesh 406. The mesh 406 allows the plates to move relative to one another but keeps them coupled together and prevents them from moving too far relative to one another. In this example, the braiding is lighter than the braiding used in the proximal portions of the elongate shaft 102. In other examples the braiding may be omitted and just a polymer tube used. The pull wires W pass through the annular layer between the inner and outer layers 404, 402 and the distal ends of the pull wires are coupled to the distal portions of the plates 408, or coupled to any desired portion of the plates. Therefore, in this example there are eight plates 408. The plates may be the same size as one another or they may vary in size relative to one another.

FIG. 4B shows the plates 408 in FIG. 4A more clearly. The plates 408 may be any elastic plate material such as a metal like stainless steel or nitinol, or the plates may be a polymer. The plates have a wide base on the proximal end and a narrower base on the distal end to form an elongated and tapered trapezoidal shape. Therefore, there is a gap that flares distally and tapers proximally between adjacent plates. This gap allows the plates to move circumferentially and longitudinally relative to one another when actuated by the pull wires thereby changing the size of the lumen or the curvature of the distal catheter tip. The proximal end of the plates is coupled to the inner coil layer of the elongate shaft previously discussed. In this example, six of the plates are the same size where the base is a size 1× and due to the taper, the tips are ½× in size. The other two plates (at the 3 o'clock and 9 o'clock positions) are the same size as one another but a larger size compared to the six smaller plates. The larger plates may have a base dimension that is 2× in size and taper down to 1× size at the tip. These dimensions are not intended to be limiting and the plate sizes may be adjusted as desired. The plates are coupled together with a super elastic mesh 406 such as a mesh formed from nitinol that can expand, contract and stretch. The mesh may return to an unbiased configuration after being expanded therefore the lumen will also return to an unbiased shape after having been expanded or having its shape changed.

The pull wires may be coupled anywhere to the plates but in this example the pull wires are coupled to a center axis of the plates at or near the distal end of the plate. The pull wire may be coupled at the proximal end of the plate, or at an intermediate point between the proximal and distal ends of the plate. The pull wires may be coupled with any technique known in the art such as welding, adhesives, other mechanical attachments, etc. Actuating the pull wires in various combinations steers the distal end of the aspiration catheter so the aspiration lumen aperture faces in a desired direction. Also, actuation of the pull wires in various combinations may expand or contract the size and shape of the aspiration lumen 410 as has been discussed previously and will be discussed further below. Additionally, any one or combination of the plates may include a score line, perforation, notch, or other surface modification (not illustrated) that helps the plates to bend preferentially along the line during increase or decrease of the lumen size or change in lumen shape or steering of the tip.

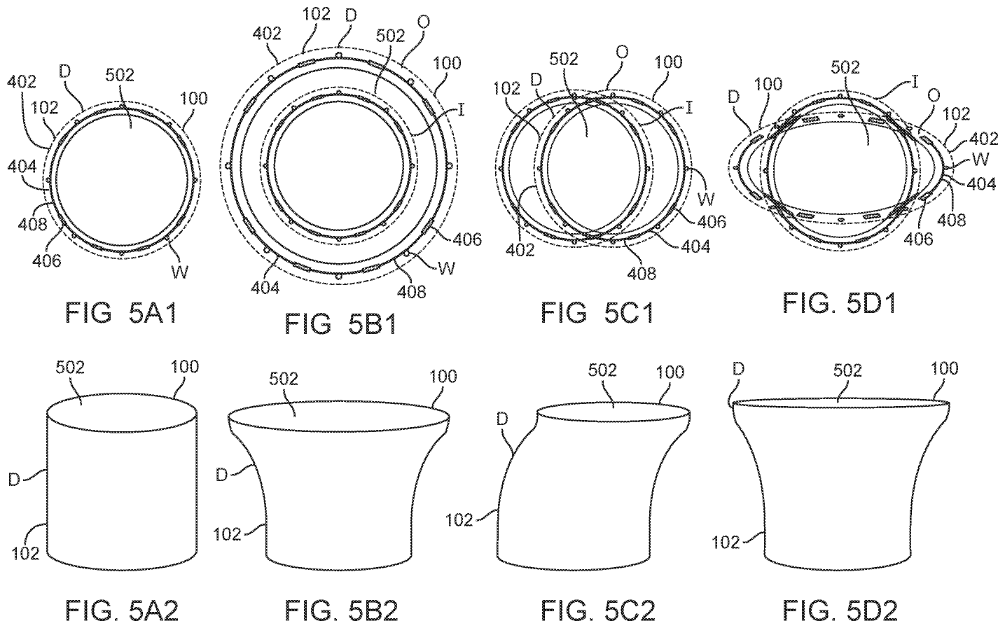

FIGS. 5A1-5D2 show various distal lumen and distal elongate shaft sizes and shapes that may be formed with the aspiration catheter of FIG. 1.

FIG. 5A1 shows the distal portion D of the elongate shaft 102 on aspiration catheter 100 in FIG. 1 in the neutral or unbiased configuration. Here, the actuators (seen in FIG. 1) on the handle on the proximal portion of the catheter are in their neutral position and therefore the pull wires W (best seen in FIGS. 2B-2G) are also in a neutral position. Thus, the distal lumen 502 shape has a circular cross-section and the size is the unbiased lumen size that is the same as the rest of the lumen size in the elongate shaft. In this example, the lumen size and shape at the distal portion of the catheter is the same as the lumen size and shape at the proximal portion of catheter as well as along intermediate portions of the elongate shaft between the proximal and distal portions of the catheter. Other cross-sectional unbiased shapes may also be employed, including but not limited to oval, square, triangular, etc. The plates 408, mesh 406, pull wires W, and inner and outer layers 404, 402 of the shaft 102 are also visible. Also, the distal portion of catheter 100 remains in its neutral shape which is a linear and cylindrical shape as seen in FIG. 5A2.

FIGS. 5B1-5B2 show another example of a configuration of the aspiration catheter which may be obtained. Here, expansion of the distal portion D of the aspiration catheter 100 from its unbiased size and shape (inner assembly I) to an expanded configuration (outer assembly O) is illustrated. The aspiration lumen 502 size is increased by actuating the proximal-most actuator as previously described in FIG. 2E where all eight pull wires W are tensioned, thereby expanding uniformly the lumen 502 size from a smaller circular cross-section to a larger diameter circular cross-section. The shape of the lumen 502 remains the same during the expansion and the shape of the distal portion of the elongate shaft also remains linear and cylindrically shaped during the expansion but the lumen size increases. FIG. 5A1 shows expansion of the lumen 502 size from the smaller unbiased diameter (seen in inner assembly I) to a larger expanded diameter (outer assembly O. FIG. 5B2 shows the expansion in the lumen size which forms a flared or trumpet shaped distal end while the remainder of the elongate shaft remains substantially linear and cylindrically shaped. The larger size of the lumen allows the aspiration catheter to more closely fit with the thrombus material to be aspirated as will be discussed further below. The plates 408, mesh 406, pull wires W, and inner and outer layers 404, 402 of the shaft 102 are also visible. Other aspects of the aspiration catheter are generally the same as previously described in FIGS. 1, 2E, and 5A1-5A2 as well as other figures in this specification.

FIG. 5C1-5C2 show another example of a configuration that may be achieved with the aspiration catheter 100 such as described in FIG. 1, by actuation of the pull wires W as described previously in FIG. 2F where the intermediate actuator is actuated. Here, actuation of the intermediate actuator does not change the lumen 502 size at the distal end of the aspiration catheter, but steers the distal end of the aspiration catheter laterally toward the right. The inner assembly I shows the catheter in the unbiased configuration, while the outer assembly O shows the catheter after actuation of the pull wires steers the catheter tip to the right. FIG. 5C1 shows the distal lumen shifted laterally to the right relative to the lumen in the main portion of the aspiration catheter which remains in the neutral or original position. FIG. 5C2 shows the steering of the distal end of the aspiration catheter laterally to the right while the remainder of the catheter maintains its original axial and linear configuration. The plates 408, mesh 406, pull wires W, and inner and outer layers 404, 402 of the shaft 102 are also visible. Other aspects of FIGS. 5C1-5C2 are generally the same as previously described in FIGS. 1, 2F, and 5A1-5A2, as well as other figures in this specification.

FIGS. 5D1-5D2 show another example of a configuration that may be achieved with the aspiration catheter 100 shown in FIG. 1. Here, actuation of the distal-most actuator as illustrated and described in FIG. 2G changes the lumen 502 shape. Actuation of the distal-most actuator applies tension to two pull wires W that are 180 degrees opposite one another which results in a change in the lumen 502 shape from the original unbiased round cross-section (inner assembly I) to an oval or elliptically shaped lumen shape (oval outer assembly O). This geometry may be accomplished without steering the catheter distal portion as seen in FIG. 5D2 where the distal end is flared outwardly on the sides to form a partial trumpet shape with the other sides moved radially inward. Thus, the distal portion is still axially oriented and cylindrical except at the tip where the lumen has become oval or elliptically shaped. In other examples, the shape change may be combined with other configurations to change shape and also steer the catheter tip. The plates 408, mesh 406, pull wires W, and inner and outer layers 404, 402 of the shaft 102 are also visible. Other aspects of FIGS. 5D1-5D2 are generally the same as previously described in FIGS. 1, 2F, and 5A1-5A2, as well as other figures in this specification.

In other examples, the pull wires and actuators may be configured to reduce the diameter of the distal aspiration catheter tip and lumen size. Actuation of one or more of the actuators may reduce the tip diameter and lumen size symmetrically or asymmetrically to match the thrombus being aspirated. Here, actuation of the actuators applies tension to one or more of the pull wires to pull them radially inward which results in the reduced diameter of the catheter tip and lumen diameter. In other examples, the pull wires may be placed in compression to help collapse the catheter tip and lumen into a smaller diameter.

As previously mentioned, other actuator/pull wire combinations are possible to effect different sizes and shape of the lumen and catheter distal portion. Therefore, the examples in FIGS. 5A1-5D2 are not intended to be limiting. Additionally, any of the examples of different sizes, shapes, and steering may be combined in any combination or permutation. Thus, the aspiration catheter tip may be steered in any direction and may have any desired geometry for aspiration of thrombus material. Moreover, one of skill in the art will appreciate that actuating any of the actuators described previously in the opposite direction generally releases tension in the actuated pull wires thereby allowing the catheter to return to its unbiased configuration.

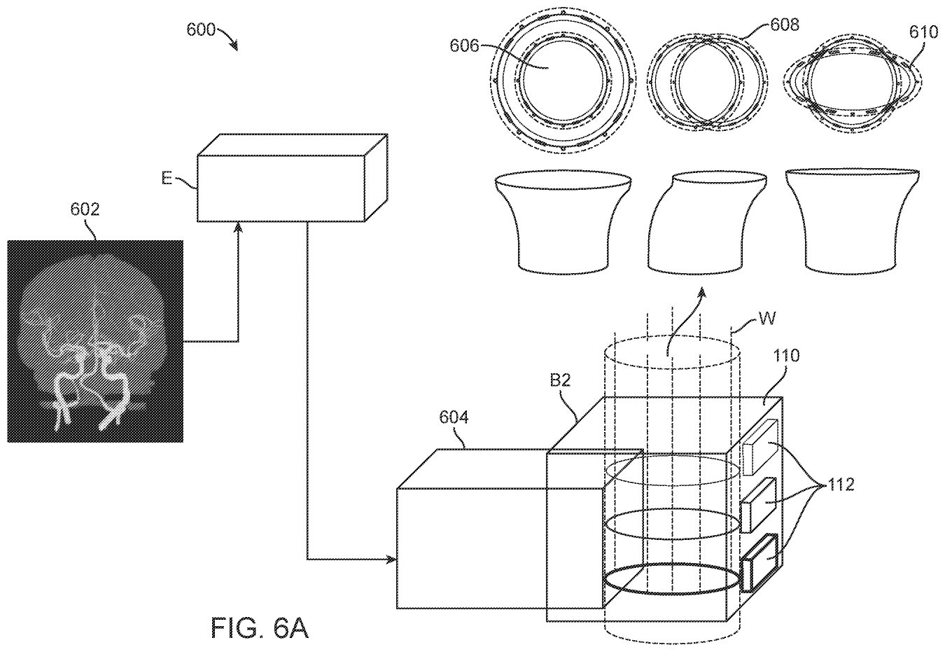

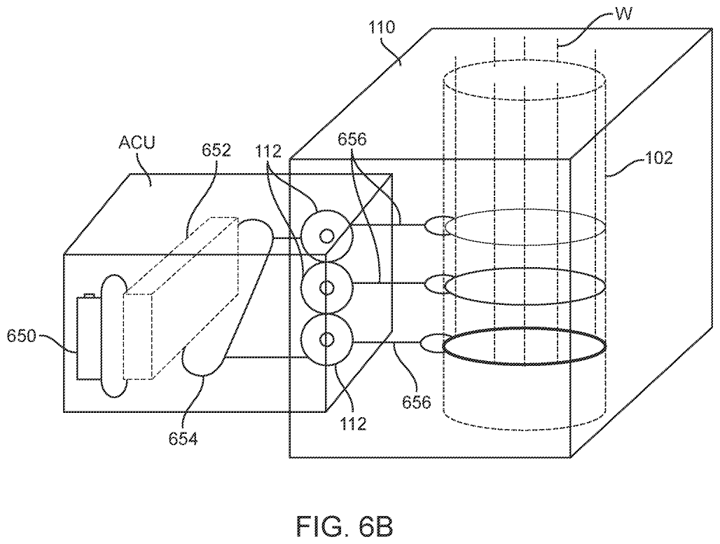

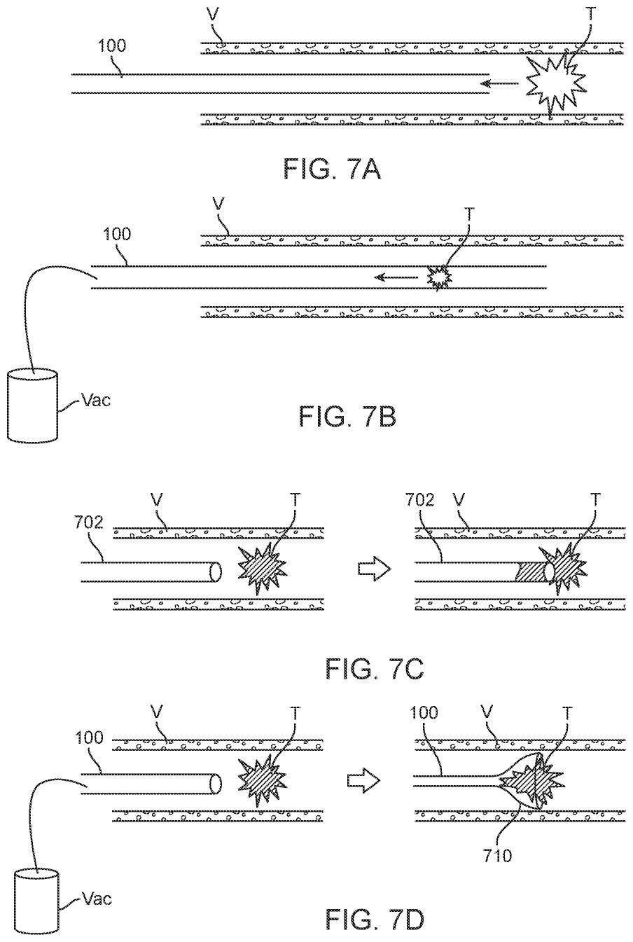

FIG. 6A shows and example of a system 600 that may be used to control any of the aspiration catheters described herein that may be used for aspiration of thrombus material from a blood vessel such as an artery or another anatomical area in a patient's body. The system uses a controller to adjust lumen size, shape, and steering of an aspiration catheter such as the catheter described in FIG. 1. Here, controller E from FIG. 1 may be provided with input from an imaging system such as a CT image or an angiogram 602 of the thrombus at the treatment site. Based on the image, the controller E is able to determine the optimal lumen size, lumen shape and distal catheter shape to characterize the thrombus being treated and consequently position the aspiration lumen of the aspiration catheter adjacent a thrombus to be aspirated from a vessel such as an artery of a patient. Based on the optimal size, shape, and distal catheter shape determined by the controller E, the controller generates commands that are then delivered to the proximal portion B1 of the aspiration catheter such as the catheter described in FIG. 1 above and the commands are then delivered to the actuators 112 on the aspiration catheter housing or handle 110 which then result in pull wires being actuated to produce the desired size, or shape of the catheter shaft or lumen. Thus, the lumen 606 size may be increased or decreased (catheter end view shows before and after actuation on top and catheter side view shown below illustrates after actuation), the distal end 608 of the aspiration catheter may be steered in any desired direction (similarly, catheter end view shows before and after actuation on top and catheter side view shown below illustrates after actuation), and/or the lumen shape 610 may be adjusted to any desired geometry (similarly catheter end view shows before and after actuation on top and catheter side view shown below illustrates after actuation). An interface 604 allows the control signals from the controller E to be transmitted to the actuators 112. The interface 604 may be a connector that is coupled with a cooperating connector on the handle 110, or it may be a wireless device for transmitting the information. Other aspects of the catheter system are generally the same as described in this specification. Once the desired size or shape has been established, the thrombus may be suctioned out of the patient's blood vessel to help restore normal blood flow as will be illustrated in FIGS. 7A-7D below.

FIG. 6B shows an example of an automated control unit (ACU) that may be fixedly or releasably coupled to the handle 110 of the aspiration catheter 100 such as the aspiration catheter illustrated in FIG. 1, and that can control actuation of the actuators 112. An optional battery 650 may be used or an external power source may be used to provide power to a processor 652 which delivers control signals to a motor 654 based on the desired shapes and sizes determined from an image of the thrombus to be treated. The control signals are sent to the motor 654 which is operably coupled 656 to the actuators 112 on the aspiration catheter handle 110. Therefore, when the motor turns in a first direction, the corresponding actuator is moved in a first direction to apply tension to the corresponding pull wire W disposed in the elongate shaft 102 of the aspiration catheter. And similarly, when the motor turns in a second direction opposite the first direction, the corresponding actuator moves in a second direction opposite its first direction and the tension is released from the corresponding pull wire, while a tension may be applied in a different pull wire. As tension is adjusted in the pull wires, the aspiration catheter lumen size, shape, and steering of the catheter will be adjusted to a desired configuration. This is not intended to be limiting and other mechanisms may be used in other examples.

FIGS. 7A-7B show an example of a method of removing thrombus from a patient using any of the examples of aspiration catheters disclosed herein.

In FIG. 7A the aspiration catheter 100 is advanced through a vessel V such as an artery toward a thrombus T. The lumen size, lumen shape and distal catheter shape or curvature is then optionally adjusted using the actuators on the aspiration catheter either manually or with the optional controller.

In FIG. 7B, vacuum Vac is applied to the aspiration catheter and the thrombus is then aspirated out of the blood vessel and removed from the patient via the lumen in the aspiration catheter as shown. The aspiration catheter may be removed once the desired amount of thrombotic material is removed from the patient.

In some examples, the ability to control the size and shape of the lumen as well as the distal catheter shape may allow the aspiration catheter to be used like a set of jaws or forceps. For example, the distal lumen could be expanded and then advanced over a proximal portion of a clot. Then the lumen diameter could be reduced pinching the clot in the process. The clot can then be retracted proximally out of the patient's vessel with or without vacuum aspiration.

FIG. 7C shows aspiration of thrombus from a vessel using a standard aspiration catheter which does not permit adjustment of the lumen size, shape, or distal catheter tip shape.

In FIG. 7C, the aspiration catheter 702 is advanced through the blood vessel V such as an artery so that the distal tip is adjacent the thrombus T. Because the aspiration catheter is not controllable, the lumen size may be smaller than the thrombus T, or the tip may not be aligned with the thrombus and thus when the vacuum is applied, not all the thrombus material may be removed, and further manual manipulation of the catheter may be required to reposition the aspiration catheter tip so that it is adjacent any remaining thrombus material. This may require multiple attempts at repositioning in order to aspirate all or a significant portion of the thrombus.

FIG. 7D illustrates how an aspiration catheter such as the examples disclosed herein which can be controlled is more easily configured to aspirate the thrombus material.

In FIG. 7D the aspiration catheter 100 which may be any of the catheters described herein is advanced through a blood vessel V such as an artery until it is adjacent the thrombus T. The distal end of the aspiration catheter may then be manually adjusted or automatically adjusted to more precisely match the thrombus being treated, using any of the examples of controllers described here. Here, the distal lumen size 710 is enlarged to more precisely approximate the size of the thrombus T so that the suction from the vacuum Vac will be applied to the entire thrombus. In other examples, the lumen shape may be adjusted to match the thrombus shape and the distal end of the catheter may be steered so that the lumen is more precisely aligned with the thrombus. Therefore, a change in size or shape of the distal lumen or curvature of the distal catheter portion better aspirates the clot. Additional details of matching the clot-catheter diameter and size are disclosed in “Tailored Vessel-Catheter Diameter Ratio in a Direct Aspiration First-Pass Technique: Is It a Matter of Caliber?” published by E. Pampana et al. in AJNR Am J Neuroradiol. 2021 March; 42(3): 546-550; the entire contents of which are incorporated herein by reference.

While the present disclosure has focused on using examples of the manipulatable catheter for aspiration of a thrombus, one of skill in the art will appreciate that this is not intended to be limiting and the examples of catheters may be used for other treatments. For example, the ability to increase or decrease the distal catheter tip size and shape and lumen size and shape may be used in the treatment of focal stenosis. Changing the catheter or lumen diameter, shape, or curvature allows the operator to manipulate the vessel wall such as by increasing its diameter, adding curvature which may allow better access to the stenosis for treatment whether by aspiration, angioplasty, stenting, drug delivery, or other treatments known in the art. Similarly, in situations where there is vasospasm, the catheter may be used to dilate the collapsed vessel into a larger more easily treatable size or shape.

### NOTES AND EXAMPLES

The following, non-limiting examples, detail certain aspects of the present subject matter to solve the challenges and provide the benefits discussed herein, among others.

Example 1 is a device for aspirating thrombus from a patient, the device comprising: an elongate shaft having a proximal portion, a distal portion, a lumen extending therebetween, and a sidewall extending therebetween; a housing coupled to the proximal portion, the housing comprising an actuator coupled thereto; a plurality of pull wires disposed in the side wall of the elongate shaft, the plurality of pull wires each having a proximal end and a distal end, wherein the proximal ends of the plurality of pull wires are coupled to the actuator, and wherein the distal ends of the plurality of pull wires are coupled to the distal portion of the elongate shaft, and wherein actuation of the actuator in a first direction applies a force to at least some of the plurality of pull wires to steer the distal portion of the elongate shaft into a first configuration or to change a size of the lumen or change a shape of the lumen adjacent the distal end of the elongate shaft, and wherein actuation of the actuator in a second direction opposite the first direction releases the applied force in the at least some of the plurality of pull wires to steer the distal portion of the elongate shaft into a second configuration or to further change the size of the lumen or change the shape of the lumen adjacent the distal end of the elongate shaft.

Example 2 is the device of Example 1, wherein the actuator comprises a plurality of actuators, each of the plurality of actuators configured to steer the distal portion of the elongate shaft into a different configuration or configured to change the size or the shape of the lumen adjacent the distal end of the elongate shaft.

Example 3 is the device of any of Examples 1-2, further comprising a plurality of lumens disposed in the side wall of the elongate shaft, the plurality of pull wires disposed in the plurality of lumens.

Example 4 is the device of any of Examples 1-3, further comprising a liner disposed in at least some of the plurality of lumens, the liner configured to reduce friction in the at least some of the plurality of lumens so as to facilitate slidable movement of the plurality of pull wires in the plurality of lumens.

Example 5 is the device of any of Examples 1-4, further comprising a connector coupled to the proximal portion of the elongate shaft, the connector configured to permit coupling of the elongate shaft with another device or anchoring to a patient.

Example 6 is the device of any of Examples 1-5, wherein at least a portion of the elongate shaft is a braided shaft.

Example 7 is the device of any of Examples 1-6, wherein at least a portion of the elongate shaft comprises a coiled filament.

Example 8 is the device of any of Examples 1-7, wherein the lumen is configured to receive thrombus, or wherein the change in the shape is configured to reduce or eliminate contraction of a blood vessel in vasospasm.

Example 9 is the device of any of Examples 1-8, further comprising a controller releasably coupled to the actuator, the controller configured to actuate the actuator.

Example 10 is the device of any of Examples 1-9, wherein the controller is configured to actuate the actuator to control the diameter or the shape of the lumen or the shape of the distal portion of the elongate shaft based on images of a target treatment vessel.

Example 11 is the device of any of Examples 1-10, wherein the distal portion comprises a plurality of plates coupled together with a mesh, the plurality of plates coupled to the plurality of pull wires and the plurality of plates configured to move relative to one another when the actuator is actuated.

Example 12 is the device of any of Examples 1-11, wherein the housing comprises a handle.

Example 13 is a method for changing a shape or a lumen size or lumen shape of a catheter, the method comprising: actuating an actuator on a handle coupled to a proximal end of an elongate shaft thereby applying a force in one or more pull wires disposed in a side wall of the elongate shaft, wherein the one or more pull wires are coupled to a distal end of the elongate shaft; and steering the distal end of the elongate shaft or changing a size or shape of a lumen in the elongate shaft adjacent the distal end of the elongate shaft as a result of the elongate shaft.

Example 14 is the method of Example 13, further comprising disposing the elongate shaft in a blood vessel and aspirating thrombus out of the vessel into the lumen, or further comprising disposing the elongate shaft in a blood vessel in vasospasm and reduce or eliminating the vasospasm.

Example 15 is the method of any of Examples 13-14, wherein the actuator comprises a plurality of actuators, and wherein actuating the actuator comprises actuating one or more of the plurality of actuators to steer the distal portion of the elongate shaft or to change the size or shape of the lumen adjacent the distal portion of the elongate shaft.

Example 16 is the method of any of Examples 13-15, wherein the actuating the actuator comprises slidably moving one or more of the plurality of pull wires through a lumen disposed in a side wall of the elongate shaft.

Example 17 is the method of any of Examples 13-16, wherein a liner is disposed in at least some of the plurality of lumens and moving the one or more plurality of pull wires comprises moving the one or more plurality of pull wires through the liner.

Example 18 is the method of any of Examples 13-17, further comprising coupling the proximal end of the elongate shaft with another device or anchoring the proximal end of the elongate shaft to a patient with a connector disposed on a proximal portion of the elongate shaft.

Example 19 is the method of any of Examples 13-18, further comprising releasably coupling a controller to the actuator, and actuating the actuator with the controller.

Example 20 is the method of any of Examples 13-19, further comprising actuating the actuator with the controller to control a size or shape of the lumen or a shape of the distal end of the elongate shaft based on images of a target treatment vessel.

Example 21 is the method of any of Examples 13-20, wherein the plurality of pull wires is coupled to a plurality of plates coupled together with a mesh, and wherein steering the distal end of the elongate shaft or changing the size or shape of the lumen comprises moving the plurality of plates relative to one another.

Example 22 is a method for removing a thrombus from a vessel in a patient, the method comprising: advancing an elongate shaft toward a clot; enlarging a lumen or changing a shape of the lumen in the elongate shaft; positioning the elongate shaft over the clot so that the clot is at least partially disposed in the lumen; reducing the lumen size or changing the lumen shape thereby capturing the clot in the lumen; and removing the clot from vessel with or without vacuum aspiration.

In Example 23, the devices, systems, or method of any one or any combination of Examples 1-22 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A device for aspirating thrombus from a patient, the device comprising:
an elongate shaft having a proximal portion, a distal portion, a lumen extending therebetween, and a sidewall extending therebetween;
a housing coupled to the proximal portion, the housing comprising an actuator coupled thereto;
a plurality of pull wires disposed in the side wall of the elongate shaft, the plurality of pull wires each having a proximal end and a distal end,
wherein the proximal ends of the plurality of pull wires are coupled to the actuator, and
wherein the distal ends of the plurality of pull wires are coupled to the distal portion of the elongate shaft, and
wherein actuation of the actuator in a first direction applies a force to at least some of the plurality of pull wires to steer the distal portion of the elongate shaft into a first configuration or to change a size of the lumen or change a shape of the lumen adjacent the distal end of the elongate shaft, and
wherein actuation of the actuator in a second direction opposite the first direction releases the applied force in the at least some of the plurality of pull wires to steer the distal portion of the elongate shaft into a second configuration or to further change the size of the lumen or change the shape of the lumen adjacent the distal end of the elongate shaft.

2. The device of claim 1, wherein the actuator comprises a plurality of actuators, each of the plurality of actuators configured to steer the distal portion of the elongate shaft into a different configuration or configured to change the size or shape of the lumen adjacent the distal end of the elongate shaft.

3. The device of claim 1, further comprising a plurality of lumens disposed in the side wall of the elongate shaft, the plurality of pull wires disposed in the plurality of lumens.

4. The device of claim 3, further comprising a liner disposed in at least some of the plurality of lumens, the liner configured to reduce friction in the at least some of the plurality of lumens so as to facilitate slidable movement of the plurality of pull wires in the plurality of lumens.

5. The device of claim 1, further comprising a connector coupled to the proximal portion of the elongate shaft, the connector configured to permit coupling of the elongate shaft with another device or anchoring to a patient.

6. The device of 1, wherein at least a portion of the elongate shaft is a braided shaft.

7. The device of claim 1, wherein at least a portion of the elongate shaft comprises a coiled filament.

8. The device of claim 1, wherein the lumen is configured to receive thrombus, or wherein the change in the shape is configured to reduce or eliminate contraction of a blood vessel in vasospasm.

9. The device of claim 1, further comprising a controller releasably coupled to the actuator, the controller configured to actuate the actuator.

10. The device of claim 9, wherein the controller is configured to actuate the actuator to control the diameter or the shape of the lumen or the shape of the distal portion of the elongate shaft based on images of a target treatment vessel.

11. The device of claim 1, wherein the distal portion comprises a plurality of plates coupled together with a mesh, the plurality of plates coupled to the plurality of pull wires and the plurality of plates configured to move relative to one another when the actuator is actuated.

12. The device of claim 1, wherein the housing comprises a handle.

13. A method for changing a shape or a lumen size or lumen shape of a catheter, the method comprising:
actuating an actuator on a handle coupled to a proximal end of an elongate shaft thereby applying a force in one or more pull wires disposed in a side wall of the elongate shaft,
wherein the one or more pull wires are coupled to a distal end of the elongate shaft; and
steering the distal end of the elongate shaft or changing a size or shape of a lumen in the elongate shaft adjacent the distal end of the elongate shaft as a result of the elongate shaft.

14. The method of claim 13, further comprising disposing the elongate shaft in a blood vessel and aspirating thrombus out of the vessel into the lumen, or further comprising disposing the elongate shaft in a blood vessel in vasospasm and reducing or eliminating the vasospasm.

15. The method of claim 13, wherein the actuator comprises a plurality of actuators, and wherein actuating the actuator comprises actuating one or more of the plurality of actuators to steer the distal portion of the elongate shaft or to change the size or the shape of the lumen adjacent the distal portion of the elongate shaft.

16. The method of claim 13, wherein the actuating the actuator comprises slidably moving one or more of the plurality of pull wires through a lumen disposed in a side wall of the elongate shaft.

17. The method of claim 16, wherein a liner is disposed in at least some of the plurality of lumens and moving the one or more plurality of pull wires comprises moving the one or more plurality of pull wires through the liner.

18. The method of claim 13, further comprising coupling the proximal end of the elongate shaft with another device or anchoring the proximal end of the elongate shaft to a patient with a connector disposed on a proximal portion of the elongate shaft.

19. The method of claim 13, further comprising releasably coupling a controller to the actuator, and actuating the actuator with the controller.

20. The method of claim 19, further comprising actuating the actuator with the controller to control a size or a shape of the lumen or a shape of the distal end of the elongate shaft based on images of a target treatment vessel.

21. The method of claim 13, wherein the plurality of pull wires is coupled to a plurality of plates coupled together with a mesh, and wherein steering the distal end of the elongate shaft or changing the size or shape of the lumen comprises moving the plurality of plates relative to one another.

22. A method for removing a thrombus from a vessel in a patient, the method comprising:
advancing an elongate shaft toward a clot;
enlarging a lumen or changing a shape of the lumen in the elongate shaft;
positioning the elongate shaft over the clot so that the clot is at least partially disposed in the lumen;
reducing the lumen size or changing the lumen shape thereby capturing the clot in the lumen; and
removing the clot from vessel with or without vacuum aspiration.
